(19) United States
(12) Patent Application Publication
(10) Pub. No.: US 2008/0207675 A1
(43) Pub. Date: Aug. 28, 2008

(54) Aqueous Gel Formulations Containing 1-(2-Methylpropyl)-1H-Imidazo[4,5-C][1,5]Naphthyridin-4-Amine

(75) Inventors: David Q. Ma, Saint Paul, MN (US); Christopher S. Perman, Saint Paul, MN (US); Raymond D. Skwierczynski, Cambridge, MA (US); John C. Hedenstrom, Saint Paul, MN (US)

Correspondence Address: FOLEY AND LARDNER LLP;SUITE 500, 3000 K STREET NW, WASHINGTON, DC, 20007, US

(73) Assignee: Takeda Pharmaceutical Company Limited

(21) Appl. No.: 11/815,491

(22) PCT Filed: Feb. 3, 2006
(86) PCT No.: PCT/US06/03745

§ 371 Date: Aug. 3, 2007

Related U.S. Application Data
(60) Provisional application No. 60/649,932, filed on Feb. 4, 2005.
(60) Provisional application No. 60/698,416, filed on Jul. 12, 2005.

(51) Int. Cl. A61K 31/437; C07D 471/12; A61P 1/04

(52) U.S. Cl. 514/293; 546/82

(57) ABSTRACT

Pharmaceutical formulations in an aqueous gel formulation including 1-(2-methylpropyl)-1H-imidazo[4,5-c][1,5]naphthyridin-4-amine are provided. Methods of use and kits are also provided.

### CROSS-REFERENCE TO RELATED APPLICATIONS

This application claims benefit of U.S. provisional application 60/649,932, filed on Feb. 4, 2005, and of U.S. provisional application 60/698,416, filed on Jul. 12, 2005, the contents of both of which are incorporated by reference herein.

### BACKGROUND

Many imidazoquinoline amine, imidazopyridine amine, 6,7-fused cycloalkylimidazopyridine amine, 1,2-bridged imidazoquinoline amine, thiazoloquinoline amine, oxazoloquinoline amine, thiazolopyridine amine, oxazolopyridine amine, imidazonaphthyridine amine, imidazotetrahydronaphthyridine amine, and thiazolonaphthyridine amine compounds have demonstrated potent immunostimulating, antiviral and antitumor (including anticancer) activity, and have also been shown to be useful as vaccine adjuvants and in the treatment of TH2-mediated diseases. These compounds are hereinafter collectively referred to as “IRM” (immune response modifier) compounds.

The mechanism for the immunostimulatory activity of these IRM compounds is thought to be due in substantial part to enhancement of the immune response by induction of various important cytokines (e.g., interferons, interleukins, tumor necrosis factor, etc.). Such compounds have been shown to stimulate a rapid release of certain monocyte/macrophage-derived cytokines and are also capable of stimulating B cells to secrete antibodies, which play an important role in these IRM compounds' activities. One of the predominant immunostimulating responses to these compounds is the induction of interferon (IFN)-α production, which is believed to be very important in the acute antiviral and antitumor activities seen, Moreover, up regulation of other cytokines such as, for example, tumor necrosis factor (TNF), Interleukin-1 (IL-1), IL-6, and IL-12 also have potentially beneficial activities and are believed to contribute to the antiviral and antitumor properties of these compounds.

Although some of the beneficial effects of IRMs are known, the ability to provide therapeutic benefit via topical application of an IRM compound for treatment of a particular condition at a particular location may be hindered by a variety of factors. These factors include irritation of the dermal or mucosal tissue to which the formulation is applied, ciliary clearance of the formulation, formulation wash away, insolubility and/or degradation of the IRM compound in the formulation, physical instability of the formulation (e.g., separation of components, thickening, precipitation/agglomeration of active ingredient, and the like), and poor permeation, for example. Accordingly, there is a continuing need for new methods and formulations to provide the greatest therapeutic benefit from this class of compounds.

### SUMMARY

The present invention is directed to aqueous gel formulations, kits, and methods of use. Herein, a “gel”, is a composition that is substantially free of oil (and hence, is not a cream or a lotion). Preferably, gels of the present invention have a viscosity of at least 1000 Centipoise (cps) at room temperature (i.e., about 20° C.). Preferably, gels of the present invention have a viscosity of no greater than 50,000 cps, and more preferably no greater than 30,000 cps.

Aqueous gels are not easily formed using certain IRMs, particularly 1-(2-methylpropyl)-1H-imidazo[4,5-c][1,5]naphthyridin-4-amine, due to the low solubility of the free base in water. As a result, a cosolvent is typically used or an IRM salt is prepared in situ. This can result in the need for negatively charged thickeners, particularly two negatively charged thickeners, to provide the desirable viscosity. In preferred embodiments of the present invention, the negatively charged thickeners are not covalently bonded to the 1-(2-methylpropyl)-1H-imidazo[4,5-c][1,5]naphthyridin-4-amine.

In one embodiment, such aqueous gels include: water; 1-(2-methylpropyl)-1H-imidazo[4,5-c][1,5]naphthyridin-4-amine; a pharmaceutically acceptable acid; at least 10 wt-% of a water-miscible cosolvent; and a thickener system including a negatively charged thickener (preferably, at least two negatively charged thickeners); wherein the aqueous gel has a viscosity of at least 1000 Centipoise (cps) at 20° C.

In one embodiment, such aqueous gels are prepared by a method that includes combining components including: water; a 1-(2-methylpropyl)-1H-imidazo[4,5-c][1,5]naphthyridin-4-amine salt; at least 10 wt-% of a water-miscible cosolvent; and a thickener system including a negatively charged thickener (preferably, at least two negatively charged thickeners); wherein the aqueous gel has a viscosity of at least 1000 cps at 20° C.

Aqueous gel formulations of the present invention can provide desirable vehicles for 1-(2-methylpropyl)-1H-imidazo[4,5-c][1,5]naphthyridin-4-amine and can allow for easier manufacture and increased residence time of 1-(2-methylpropyl)-1H-imidazo[4,5-c][1,5]naphthyridin-4-amine, particularly on mucosal tissue.

Furthermore, the use of negatively charged thickeners in the aqueous gels of the present invention reduces systemic exposure to the drug and hence reduces systemic levels of cytokines. This is desirable for many conditions for which treatment at a particular location (e.g., cervical dysplasia) is preferred. The use of a combination of negatively charged thickeners (i.e., at least two) is desirable when higher levels of cosolvents are used due to the low solubility of the drug (whether in free base or salt form) in water. This results in an aqueous gel that reduces systemic exposure and is physically stable.

The present invention also provides methods of using the formulations of the present invention. In one embodiment, the present invention provides a method for delivering 1-(2-methylpropyl)-1H-imidazo [4,5-c][1,5]naphthyridin-4-amine to the mucosal tissue of a subject, the method including applying an aqueous gel of the present invention. Preferably, the mucosal tissue is associated with a condition selected from the group consisting of a cervical dysplasia, a papilloma virus infection of the cervix, a low-grade squamous intraepithelial lesion, a high-grade squamous intraepithelial lesion, atypical squamous cells of undetermined significance, a cervical intraepithelial neoplasia, an atopic allergic response, allergic rhinitis, a neoplastic lesion, and a premalignant lesion.

The present invention also provides kits that include a barrel type applicator and an aqueous gel of the present invention, which can be in a separate container or prefilled in the barrel type applicator.

The terms “comprises” and variations thereof do not have a limiting meaning where these terms appear in the description and claims.

As used herein, “a,” “an,” “the,” “at least one,” and “one or more” are used interchangeably. Thus, for example, an aqueous gel that comprises “a” preservative can be interpreted to mean that the gel includes “one or more” preservatives.

Also herein, the recitations of numerical ranges by endpoints include all numbers subsumed within that range (e.g., 1 to 5 includes 1, 1.5, 2, 2.75, 3, 3.80, 4, 5, etc.).

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE DRAWINGS

FIG. 1 is a representative X-ray diffraction pattern of a crystalline form of 1-(2-methylpropyl)-1H-imidazo[4,5-c][1,5]naphthyridin-4-amine ethanesulfonate monohydrate.

FIG. 2 is a representative solid state ¹³C NMR spectrum of a crystalline form of 1-(2-methylpropyl)-1H-imidazo[4,5-c][1,5]naphthyridin-4-amine ethanesulfonate monohydrate.

FIG. 3 is a representative water sorption isotherm curve for a crystalline form of 1-(2-methylpropyl)-1H-imidazo[4,5-c][1,5]naphthyridin-4-amine ethanesulfonate monohydrate.

FIG. 4 is a representative thermogram of a crystalline form of 1-(2-methylpropyl)-1H-imidazo[4,5-c][1,5]naphthyridin-4-amine ethanesulfonate monohydrate, which shows an overlay of data obtained by DSC and TGA.

### DETAILED DESCRIPTION OF ILLUSTRATIVE EMBODIMENTS

The present invention provides aqueous gel formulations, kits, and methods of use. Such gels are compositions that are substantially free of oil (and hence, they are not creams or lotions). Preferably, gels of the present invention have a viscosity of at least 1000 Centipoise (cps) at 20° C. Preferably, gels of the present invention have a viscosity of no greater than 50,000 cps, and more preferably no greater than 30,000 cps.

In one embodiment, such aqueous gels include: water; 1-(2-methylpropyl)-1H-imidazo[4,5-c][1,5]naphthyridin-4-amine; a pharmaceutically acceptable acid; at least 10 wt-% of a water-miscible cosolvent; and a thickener system including a negatively charged thickener (preferably, at least two negatively charged thickeners, which are typically of different charge density); wherein the aqueous gel has a viscosity of at least 1000 Centipoise (cps) at 20° C.

In one embodiment, such aqueous gels are prepared by a method that includes combining components including: water; a 1-(2-methylpropyl)-1H-imidazo[4,5-c][1,5]naphthyridin-4-amine salt; at least 10 wt-% of a water-miscible cosolvent; and a thickener system including a negatively charged thickener (preferably, at least two negatively charged thickeners, which are typically of different charge density); wherein the aqueous gel has a viscosity of at least 1000 cps at 20° C.

The immune response modifier is substantially completely dissolved at a therapeutic level (i.e., therapeutically effective amount) in the formulation at room temperature. This amount is effective to treat and/or prevent a specified condition. In general, the amount of 1-(2-methylpropyl)-1H-imidazo[4,5-c][1,5]naphthyridin-4-amine present in an aqueous gel formulation of the invention will be an amount effective to provide a desired physiological effect, e.g., to treat a targeted condition, to prevent recurrence of the condition, or to promote immunity against the condition. For certain embodiments, an amount effective to treat or inhibit a viral infection is an amount that will cause a reduction in one or more manifestations of viral infections, such as viral load, rate of virus production, or mortality as compared to untreated control animals.

In certain methods of the present invention, the mucosal tissue is associated with a condition selected from the group consisting of a cervical dysplasia, a papilloma virus infection of the cervix, a low-grade squamous intraepithelial lesion, a high-grade squamous intraepithelial lesion, atypical squamous cells of undetermined significance, a cervical intraepithelial neoplasia, an atopic allergic response, allergic rhinitis, a neoplastic lesion, and a premalignant lesion.

In certain methods of the present invention, the mucosal tissue is on the cervix and the associated condition is selected from the group consisting of cervical dysplasia, high-grade squamous intraepithelial lesions, low-grade squamous intraepithelial lesions, and atypical squamous cells of undetermined significance with the presence of high risk HPV.

In certain methods of the present invention, the mucosal tissue is on the cervix and the associated condition is atypical squamous cells of undetermined significance with the presence of high risk HPV.

In certain methods of the present invention, the mucosal tissue is on the cervix and the associated condition is a papilloma virus infection of the cervix.

The amount of 1-(2-methylpropyl)-1H-imidazo[4,5-c][1,5]naphthyridin-4-amine that will be therapeutically effective in a specific situation will depend on such things as the dosing regimen, the application site, the particular formulation and the condition being treated. As such, it is generally not practical to identify specific administration amounts herein; however, those skilled in the art will be able to determine appropriate therapeutically effective amounts based on the guidance provided herein, information available in the art pertaining to IRMs, and routine testing.

In some embodiments, the methods of the present invention include administering sufficient formulation to provide a dose of 1-(2-methylpropyl)-1H-imidazo[4,5-c][1,5]naphthyridin-4-amine of, for example, from 100 ng/kg to 50 mg/kg to the subject, although in some embodiments the methods may be performed by administering 1-(2-methylpropyl)-1H-imidazo[4,5-c][1,5]naphthyridin-4-amine in concentrations outside this range. In some of these embodiments, the method includes administering sufficient formulation to provide a dose of 1-(2-methylpropyl)-1H-imidazo[4,5-c][1,5]naphthyridin-4-amine of from 10 μg/kg to 5 mg/kg to the subject, for example, a dose of from 100 μg/kg to 1 mg/kg.

In certain embodiments of the formulations of the invention, the amount or concentration of 1-(2-methylpropyl)-1H-imidazo[4,5-c][1,5]naphthyridin-4-amine is at least 0.0001% by weight (wt-%), in other embodiments, at least 0.001 wt-%, in other embodiments at least 0.01 wt-%, and in other embodiments at least 0.1 wt-%, based on the total weight of the aqueous gel. In certain embodiments, the amount of 1-(2-methylpropyl)-1H-imidazo[4,5-c][1,5]naphthyridin-4-amine is no greater than 7 wt-%, in other embodiments no greater than 5 wt-%, in other embodiments no greater than 3 wt-%, in other embodiments no greater than 2 wt-%, and in other embodiments no greater than 1 wt-%, based on the total weight of the aqueous gel.

1-(2-Methylpropyl)-1H-imidazo[4,5-c][1,5]naphthyridin-4-amine may be present in the formulation as the sole therapeutically active ingredient or in combination with other therapeutic agents. Such other therapeutic agents may include, for example, antibiotics, such as penicillin or tetracycline, corticosteroids, such as hydrocortisone or betamethasone, nonsteroidal antiinflammatories, such as fluriprofen, ibuprofen, or naproxen, or antivirals, such as acyclovir or valcyclovir.

In some embodiments, the above-described formulations are particularly advantageous for application for a period of time sufficient to obtain a desired therapeutic effect without undesired systemic absorption of 1-(2-methylpropyl)-1H-imidazo[4,5-c][1,5]naphthyridin-4-amine.

The IRM of the present invention, 1-(2-methylpropyl)-1H-imidazo[4,5-c][1,5]naphthyridin-4-amine, is present in the gel formulations in combination with a pharmaceutically acceptable acid. Such acid is preferably present in a stoichiometric amount relative to 1-(2-methylpropyl)-1H-imidazo[4,5-c][1,5]naphthyridin-4-amine.

A wide range of pharmaceutically acceptable acids can be used to form salts of 1-(2-methylpropyl)-1H-imidazo[4,5-c][1,5]naphthyridin-4-amine. Examples of such acids are described in Berge et al., J. Pharm. Sciences, 66, 1-19 (1977). Preferred pharmaceutically acceptable acids (e.g., suitable for incorporation in the gels of the present invention or for forming salts of the IRM of the present invention) include, for example, an alkylsulfonic acid, a carboxylic acid, a halo acid, sulfuric acid, phosphoric acid, a dicarboxylic acid, a tricarboxylic acid, and combinations thereof. More preferred pharmaceutically acceptable acids include acetic acid, hydrobromic acid, D-gluconic acid, L-lactic acid, methanesulfonic acid, ethanesulfonic acid, propionic acid, and combinations thereof. Particularly preferred salts of 1-(2-methylpropyl)-1H-imidazo[4,5-c][1,5]naphthyridin-4-amine are alkylsulfonate salts (e.g., ethanesulfonate or methanesulfonate). The salt can be prepared in situ in the gel formulation. Alternatively, the salt can be prepared and isolated using conventional methods prior to being incorporated into a gel formulation.

1-(2-Methylpropyl)-1H-imidazo[4,5-c][1,5]naphthyridin-4-amine and salts thereof described herein include any of their pharmaceutically acceptable forms, such as isomers (e.g., diastereomers and enantiomers), solvates, polymorphs, and the like. In particular, if a salt is optically active, the invention specifically includes the use of each of the salt's enantiomers as well as racemic combinations of the enantiomers.

### Cosolvents

Aqueous gel formulations of the invention include a water-miscible cosolvent. The water-miscible cosolvent assists in dissolving the immune response modifier in salt form. The cosolvent can be a single component or a combination. Examples of suitable cosolvents include monopropylene glycol, dipropylene glycol, hexylene glycol, butylene glycol, glycerin, polyethylene glycol (of various molecular weights, e.g., 300 or 400), diethylene glycol monoethyl ether, and combinations thereof. Monopropylene glycol (i.e., propylene glycol) is particularly preferred as a cosolvent.

In certain embodiments, the cosolvent (or combination of cosolvents) is present in an amount of at least 10 wt-%, in other embodiments in an amount of greater than 25 wt-%, and in other embodiments at least 30 wt-%, based on the total weight of the aqueous gel. In certain embodiments, the cosolvent (or combination of cosolvents) is present in an amount of no greater than 90 wt-%, in other embodiments no greater than 80 wt-%, in other embodiments no greater than 70 wt-%, in other embodiments no greater than 60 wt-%, based on the total weight of the aqueous gel.

In certain embodiments, water is present in an amount of at least 10 wt-%, in other embodiments at least 15 wt-%, in other embodiments at least 20 wt-%, and in other embodiments at least 25 wt-%, based on the total weight of the aqueous gel. In certain embodiments, water is present in an amount of no greater than 95 wt-%, in other embodiments no greater than 90 wt-%, and in other embodiments no greater than 85 wt-%, based on the total weight of the aqueous gel.

### Negatively Charged Thickeners

Aqueous gel formulations of the invention include a negatively charged thickener, and preferably at least two negatively charged thickeners (typically of differing charge density). Preferably the thickeners are mucoadhesives. Examples of suitable negatively charged thickeners include: cellulose ethers such as carboxymethylcellulose sodium; polysaccharide gums such as xanthan gum; and acrylic acid polymers (i.e., homopolymers and copolymers) made from acrylic acid crosslinked with, for example, allyl sucrose or allyl pentaerythritol such as those polymers designated as carbomers in the United States Pharmacopoeia, and acrylic acid polymers made from acrylic acid crosslinked with divinyl glycol such as those polymers designated as polycarbophils in the United States Pharmacopoeia. Combinations of such thickeners can be used if desired.

In some embodiments of the invention, the negatively charged thickeners include carboxylic acid and/or carboxylate groups. Examples of such agents include carboxymethylcellulose sodium, xanthan gum, and the acrylic acid polymers. Preferably, certain embodiments of the present invention include a combination of an acrylic acid polymer (i.e., polyacrylic acid polymer) and a polysaccharide gum (e.g., xanthan gum).

Carbomers are exemplary (and preferred) acrylic acid polymers. Suitable carbomers include, for example, those commercially available under the trade designation CARBOPOL (all available from Noveon, Inc., Cleveland, Ohio, USA). CARBOPOL polymers can provide a range of viscosities. For example, a 0.5% solution of CARBOPOL 971P or CARBOPOL 941 has a viscosity of 4,000-11,000 cPs (pH 7.5, 25° C., Brookfield viscometer at 20 rpm); a 0.5% solution of CARBOPOL 934P or CARBOPOL 974P has a viscosity of 29,400-39,400 cPs (pH 7.5, 25° C., Brookfield viscometer at 20 rpm); and a 0.5% solution of CARBOPOL 940 or CARBOPOL 980 has a viscosity of 40,000-60,000 cPs (pH 7.5, 25° C., Brookfield viscometer at 20 rpm). For certain embodiments, carbomers such as CARBOPOL 934P, CARBOPOL 974P, CARBOPOL 940, and CARBOPOL 980 are preferred. A particularly preferred carbomer is CARBOPOL 974P.

For certain embodiments, it is desirable to have a relatively highly crosslinked carbomer. Preferred relatively highly crosslinked carbomers include CARBOPOL 974P, CARBOPOL 940, and CARBOPOL 980. A particularly preferred relatively highly crosslinked carbomer is CARBOPOL 974P.

Suitable polycarbophils include, for example, those commercially available under the trade designation NOVEON polycarbophils (all available from Noveon, Inc., Cleveland, Ohio, USA). A preferred polycarbophil is NOVEON AA-1 USP Polycarbophil.

Various grades of carboxymethylcellulose sodium are commercially available that have differing aqueous viscosities. Aqueous 1% weight by volume (w/v) solutions with viscosities of 5-13,000 cps may be obtained. Examples include carboxymethylcellulose sodium, high viscosity, USP (CA194); carboxymethylcellulose sodium, medium viscosity, USP (CA192); and carboxymethylcellulose sodium, low viscosity, USP (CA193); all of which are available from Spectrum Chemicals and Laboratory Products, Inc., Gardena, Calif., USA; and AKUCELL AF 3085 (high viscosity), AKUCELL AF 2785 (medium viscosity), and AKUCELL AF 0305 (low viscosity), all of which are available from Akzo Nobel Functional Chemicals, Amersfoort, The Netherlands.

In certain embodiments, the thickener system includes a polysaccharide gum and an acrylic acid polymer. Preferably, the weight ratio of polysaccharide gum to acrylic acid polymer is within a range of 1:20 to 20:1. In certain embodiments, the weight ratio is within a range of 1:10 to 10:1, in other embodiments the weight ratio is within a range of 1:5 to 5:1, in other embodiments the weight ratio is within a range of 1:3 to 3:1, and in other embodiments the weight ratio is within a range of 1:2 to 2:1. A particularly preferred ratio is 1:2.

The thickener system is present in formulations of the invention in an amount sufficient to bring the viscosity to a level of at least 1000 Centipoise (cps), preferably at least 5000 cps, more preferably at least 8000 cps, and most preferably at least 10000 cps. The viscosity is determined at 20±0.5° C. using a Haake RS series rheometer equipped with a 35 mm 2° cone using a controlled rate step test between 1 and 80 s⁻¹ with an interpolation at 16 s⁻¹ for viscosity versus shear rate. The values reported in the Examples below are the values at 16 s⁻¹.

In certain embodiments, the amount or concentration of the thickener system is at least 0.1 wt-%, in other embodiments at least 0.5 wt-%, in other embodiments at least 1.0 wt-%, and in other embodiments at least 1.5 wt-%, based on the total weight of the aqueous gel. In certain embodiments, the amount of the thickener system is no greater than 7 wt-%, in other embodiments no greater than 6 wt-%, in other embodiments no greater than 5 wt-%, and in other embodiments no greater than 4 wt-%, based on the total weight of the aqueous gel.

### pH Adjusting Agents and Buffers

Aqueous gel formulations of the invention can additionally include a pharmaceutically acceptable pH adjusting agent to adjust the pH of the formulation to the desired range. Generally, the pH is at least 2, and preferably at least 3. Generally, the pH is no greater than 6, preferably no greater than 5, and more preferably no greater than 4. The pH adjusting agent may be any pharmaceutically acceptable acid or base. Examples of suitable pH adjusting agents include hydrochloric acid, sodium hydroxide, tromethamine, and potassium hydroxide. Combinations of such agents can be used if desired.

Aqueous gel formulations of the invention can additionally include a pharmaceutically acceptable buffer to maintain the pH of the formulations in the desired range (preferably, 2 to 6, and more preferably, 3 to 4). The buffer may be any pharmaceutically acceptable buffer that provides one or more of the desired pH ranges. Examples of suitable buffers include buffers containing lactic acid, tartaric acid, citric acid, and succinic acid. Combinations of buffers can be used if desired. The buffers can also function as tonicity adjusting agents.

### Preservatives

Aqueous gel formulations of the invention can additionally include a preservative. The preservative includes one or more compounds that inhibit microbial growth (e.g., fungal and bacterial growth) within the composition. Suitable preservatives are water soluble and include quaternary ammonium compounds (e.g., benzalkonium chloride), benzethonium chloride, parabens (e.g., methylparaben, propylparaben), boric acid, formaldehyde donors (e.g., hydantoins), isothiazolinone, organic acids (e.g., sorbic acid), alcohols (e.g., phenyl ethyl alcohol, cresol, chlorobutanol, benzyl alcohol), carbamates, chlorhexidine, and combinations thereof. Preferably, the preservative is methylparaben, propylparaben, or combinations thereof. Certain water-miscible cosolvents, such as glycerin or propylene glycol, also have antimicrobial properties. In certain embodiments, the preservative (or combination of preservatives) is present in an amount of at least 0.005 wt-%, in other embodiments at least 0.01 wt-%, in other embodiments at least 0.015 wt-%, and in other embodiments at least 0.02 wt-%, based on the total weight of the aqueous gel. In certain embodiments, the preservative (or combination of preservatives) is present in an amount of no greater than 1.0 wt-%, in other embodiments at most 0.75 wt-%, in other embodiments at most 0.5 wt-%, and in other embodiments no greater than 0.4 wt-%, based on the total weight of the aqueous gel.

### Chelating Agents

Aqueous gel formulations of the invention can additionally include a chelating agent. Chelating agents are compounds that complex metal ions. Examples of suitable chelating agents include ethylenediaminetetracetic acid (EDTA) and derivatives thereof such as the disodium salt, ethylenediaminetetracetic acid disodium salt dihydrate, and combinations thereof. Preferably, the chelating agent is ethylenediaminetetracetic acid disodium salt dihydrate (edetate disodium).

In certain embodiments, the chelating agent (or combination of chelating agents) is present in an amount of at least 0.001 wt-%, in other embodiments at least 0.01 wt-%, and in other embodiments at least 0.02 wt-%, based on the total weight of the aqueous gel. In certain embodiments, the chelating agent (or combination of chelating agents) is present in an amount of no greater than 2.0 wt-%, in other embodiments no greater than 1.5 wt-%, and in other embodiments no greater than 1.0 wt-%, based on the total weight of the aqueous gel.

### Applications

Aqueous gel formulations of the present invention can be used to treat or prevent conditions associated with mucosal tissue. In some embodiments, the invention provides methods that are particularly advantageous for the topical application to the cervix for treatment of cervical conditions such as cervical dysplasias including dysplasia associated with human papillomavirus (HPV), low-grade squamous intraepithelial lesions, high-grade squamous intraepithelial lesions, atypical squamous cells of undetermined significance (typically, with the presence of high-risk HPV), and cervical intraepithelial neoplasia (CIN).

The present invention also provides methods of treating a mucosal associated condition. Alternatively stated, the present invention provides methods of treating a condition associated with mucosal tissue.

In the methods of the present invention, the aqueous gels of the present invention may be applied once a week or several times a week. For example, the aqueous gel may be applied twice a week, three times a week, five times a week, or even daily.

In the methods of the present invention, the applications of the aqueous gels of the present invention may extend for a total time period of at least one week, at least two weeks, at least three weeks, at least one month, at least two months, at least three months, or more, depending on the desired treatment regimen.

The actual dosing (treatment) regimen used for a given condition or subject may depend at least in part on many factors known in the art, including, but not limited to, the physical and chemical nature of 1-(2-methylpropyl)-1H-imidazo[4,5-c][1,5]naphthyridin-4-amine, the nature of the delivery material, the amount of 1-(2-methylpropyl)-1H-imidazo[4,5-c][1,5]naphthyridin-4-amine being administered, the state of the subject's immune system (e.g., suppressed, compromised, stimulated), the method of administering 1-(2-methylpropyl)-1H-imidazo[4,5-c][1,5]naphthyridin-4-amine, and the species to which 1-(2-methylpropyl)-1H-imidazo[4,5-c][1,5]naphthyridin-4-amine is being administered.

The methods of the present invention may be applicable for any suitable subject. Suitable subjects include, but are not limited to, animals such as, but not limited to, humans, non-human primates, rodents, dogs, cats, horses, pigs, sheep, goats, cows, or birds.

The methods of the present invention are suitable for a variety of medical objectives, including therapeutic, prophylactic (e.g., as a vaccine adjuvant), or diagnostic. As used herein, “treating” a condition or a subject includes therapeutic, prophylactic, and diagnostic treatments.

The term “an effective amount” (e.g., therapeutically or prophylactically) means an amount of the compound sufficient to induce a desired (e.g., therapeutic or prophylactic) effect, such as cytokine induction, inhibition of TH2 immune response, antiviral or antitumor activity, reduction or elimination of neoplastic cells. The amount of 1-(2-methylpropyl)-1H-imidazo[4,5-c][1,5]naphthyridin-4-amine that will be therapeutically effective in a specific situation will depend on such things as the dosing regimen, the application site, the particular formulation and the condition being treated. As such, it is generally not practical to identify specific administration amounts herein; however, those skilled in the art will be able to determine appropriate therapeutically effective amounts based on the guidance provided herein and information available in the art pertaining to IRMs.

The methods of the present invention may be used for the application of 1-(2-methylpropyl)-1H-imidazo[4,5-c][1,5]naphthyridin-4-amine to mucosal tissue for the treatment of a mucosal associated condition.

As used herein, a “mucosal associated condition” means an inflammatory, infectious, neoplastic, or other condition that involves mucosal tissue or that is in sufficient proximity to mucosal tissue to be affected by a therapeutic agent topically applied to the mucosal tissue. Examples of such conditions include a papilloma virus infection of the cervix, cervical dysplasias including dysplasia associated with human papillomavirus (HPV), low-grade squamous intraepithelial lesions, high-grade squamous intraepithelial lesions, atypical squamous cells of undetermined significance (typically, with the presence of high risk HPV), and cervical intraepithelial neoplasia, an atopic allergic response, allergic rhinitis, a neoplastic lesion, and a premalignant lesion.

As used herein, “mucosal tissue” includes mucosal membranes such as buccal, gingival, nasal, ocular, tracheal, bronchial, gastrointestinal, rectal, urethral, ureteral, vaginal, cervical, and uterine mucosal membranes. For example, one could treat oral lesions, vaginal lesions, or anal lesions by the methods described. One could also use the methods in combination with mucosal application of vaccines.

In one embodiment, 1-(2-methylpropyl)-1H-imidazo[4,5-c][1,5]naphthyridin-4-amine can be applied to vaginal or supravaginal mucosal tissue for the treatment of a cervical dysplasia. In other embodiments, an IRM can be applied to the mucosal tissue of the rectum for the treatment of, e.g., anal canal condyloma.

Cervical dysplasias to be treated by the methods of the present invention preferably include dysplastic conditions such as low-grade squamous intraepithelial lesions, high-grade squamous intraepithelial lesions, atypical squamous cells of undetermined significance (typically, with the presence of high-risk HPV), and cervical intraepithelial neoplasia (CIN).

Approximately 16,000 new cases of invasive cancer of the cervix are diagnosed each year in the U.S. despite extensive screening of women to detect predictive cellular changes. There are also about 3,000 deaths due to cervical cancer in the U.S. alone and this is usually secondary to not detecting the primary cancerous lesion in a timely manner.

The Papanicoulaou Test (Pap smear) is the screening test that has been accepted since the 1950s as the method to detect abnormal cells of the cervix, including inflammation and dysplasia, which includes cervical cancer. This screening test has been widely adopted in industrialized countries and has had a profound impact on mortality associated with cervical cancers. An abnormal Pap smear prompts close observation for disease progression with the potential for the therapeutic interventions of destruction or excision of cancerous or pre-cancerous tissues. These excisional treatments are expensive, uncomfortable and associated with failure rates that range from 2% to 23% and with higher failure rates reported for the more advanced lesions. Failure rates have recently been documented to approximate 10% following laser treatment.

The etiologic agent for cervical cancer was originally thought to be the herpes virus. However, there was a gradual shift from this focus on herpes virus to the human papillomavirus (HPV). Improved experimental methods over the recent past have allowed the characterization of a full spectrum of HPV subtypes, which has resulted in the conclusion that the high risk HPV types (e.g., HPV 16, 18, and less frequently 31, 33, 35, 45) are very likely the exclusive initiating factor (i.e., oncogenic agent) for cervical dysplasia and subsequent cancers. The mechanism of HPV transformation of the normal cell to a dysplastic cell is associated with the HPV encoded oncoproteins (E6 and E7) from the high risk genotypes binding the cell's tumor suppressor gene products p53 and Rb resulting in disruption of the cell cycle control mechanism in which p53 and Rb play an important role. In addition, the application of these molecular methods has resulted in the epidemiologic observation that HPV is isolated from approximately 93% of cervical tumors, which has further strengthened the generally accepted conclusion that HPV infection is the most important initiating agent for cervical cancer.

Exposure to HPV is common in sexually active women, but it does not invariably lead to dysplasia or cancer in most of the exposed women. Infected women who harbor persistent viral DNA have about five times the chance of persistent dysplasia compared to women who are able to eradicate the virus. The importance of cell-mediated immune response to HPV infection is illustrated by the observation that the antibody mediated immune response is not effective in eliminating established infections as is demonstrated by the fact that patients with invasive cervical cancer often exhibit high antibody levels against the viral E6 and E7 proteins. This particular antibody response probably reflects extensive antigen exposure in the face of increasing tumor burden. In contrast to the apparently inconsequential effect of the humoral immune response; the cell-mediated immune response (Th-1-Type Response) appears to be effective in controlling tumor progression. Regression of intraepithelial lesions is accompanied by a cellular infiltrate consisting of CD4⁺ T-cells, CD8⁺ T-cells, natural killer cells (NK) and macrophages. This inflammatory infiltrate was usually associated with tumor regression that is in contrast to women who lack the ability to mount this inflammatory response and who experience disease progression. In addition, patients with a defect in cell-mediated immunity have increased cervical cancer rates, whereas those with defects in the production of antibody do not exhibit the same susceptibility.

Aqueous gels of the present invention may be applied to mucosal tissue with the use of a delivery device. Suitable devices include barrel type applicators, cervical caps, diaphragms, and solid matrices such as tampons, cotton sponges, cotton swabs, foam sponges, and suppositories.

In some embodiments the device can be used in combination with an aqueous gel formulation. In one embodiment, a gel containing 1-(2-methylpropyl)-1H-imidazo[4,5-c][1,5]naphthyridin-4-amine can be placed into the concave region of a cervical cap, which is then place directly over the cervix. In another embodiment, a cotton or foam sponge can be used in combination with an aqueous gel of the present invention.

In some embodiments, an applicator may be used to place the device and/or the gel in the proper location on the mucosal tissue. Examples of such applicators include, for example, paperboard or plastic tube applicators commonly used for inserting tampons or suppositories. A preferred applicator is a barrel type applicator, which may be prefilled or supplied in a in together with a container of the gel and filled by the patient.

### EXAMPLES

The following examples have been selected merely to further illustrate features, advantages, and other details of the invention. It is to be expressly understood, however, that while the examples serve this purpose, the particular materials and amounts used as well as other conditions and details are not to be construed in a matter that would unduly limit the scope of this invention.

### Preparation of 1-(2-Methylpropyl)-1H-imidazo[4,5-c][1,5]naphthyridin-4-amine ethanesulfonate monohydrate

### Part A

Under an argon atmosphere, 1,5-naphthyridin-4-ol (1.6 kg, 11 moles (mol)) was added in portions of 160 grams (g) with continuous stirring to fuming nitric acid (16 L) while maintaining the reaction temperature at 45.5° C. or below. After the addition, the reaction was stirred for 23 minutes at about 45° C., heated to reflux over a period of 2.25 hours, heated at reflux (90° C. to 95° C.) for five hours, and allowed to cool to room temperature overnight. The reaction mixture was then cooled to 7.5° C., and water (16 L) was slowly added while maintaining the reaction temperature below 25° C. The resulting mixture was cooled to 9° C., and ammonium hydroxide (20 L) was slowly added to adjust the mixture to pH 6.2 while maintaining the temperature below 15° C. The resulting mixture was stirred for ten minutes and cooled to 2.8° C. The resulting solid was isolated by filtration, washed with cold water (2×2.2 L, 4° C.), dried under vacuum at 25° C., and dried under vacuum at 75° C. for 47 hours to provide 1.778 kg of 3-nitro[1,5]naphthyridin-4-ol.

### Part B

Under an argon atmosphere, a solution of 3-nitro[1,5]naphthyridin-4-ol (1.778 kg, 9.30 mol) in N,N-dimethylformamide (DMF) (16 L) was stirred for 45 minutes at 17° C. Phosphorous oxychloride (2.095 kg, 13.7 mol) was added slowly while maintaining the temperature at about 20° C., and then the reaction was stirred for 15.25 hours at 20° C. With continuous stirring, the reaction mixture was then added over a period of 55 minutes to water (76 L) that had been cooled to 4.5° C. During the addition, the temperature of the mixture was not allowed to exceed 10° C., and the temperature was 9.5° C. at the end of the addition. The mixture resulting from the addition was stirred for 100 minutes while cooling from 9.5° C. to 2.5° C. A solid formed and was isolated by filtration, washed with water (2×8 L), and dried with suction to provide 3.3 kg of 4-chloro-3-nitro [1,5]naphthyridine.

### Part C

A solution of the material from Part B in dichloromethane (26 L) was heated to 31° C., and sodium sulfate (2 kg) and magnesium sulfate (500 g) were added. The resulting mixture was stirred for one hour and then filtered. The filter cake was washed with dichloromethane (5 L), and the filtrate was transferred to another vessel with additional dichloromethane (8 L). Under an argon atmosphere and with continuous stirring, isobutylamine (2.5 L) was added to the filtrate while maintaining a reaction temperature of 17° C. to 24° C. The reaction was stirred for 13.5 hours at a temperature of 17° C. to 24° C. and then concentrated to dryness under reduced pressure at 40° C. The resulting solid was mixed with water (18 L), and the resulting mixture was stirred at 20° C. to 21° C. for three hours and then filtered. The isolated solid was washed with water (3×3 L), pulled dry under vacuum, and further dried under vacuum for 16.5 hours at 75° C. to provide 1.98 kg of N⁴-(2-methylpropyl)-3-nitro[1,5]naphthyridin-4-amine. The product was split into five equal portions.

### Part D

A Parr vessel was charged with toluene (3.86 L), 2-propanol (386 mL), N⁴-(2-methylpropyl)-3-nitro[1,5]naphthyridin-4-amine (386 g, 1.56 mol), and 5% platinum on carbon (77.2 g, 50% w/w (weight percent) in water). The vessel was sealed and purged three times with nitrogen while the reaction mixture was stirred. The reaction mixture was then placed under hydrogen pressure (2.1×10⁵ Pa to 4.1×10⁵ Pa, 30 psi to 60 psi) for 130 minutes while maintaining the temperature between 18° C. and 22° C. This reaction was repeated four more times with reaction times ranging from 120 minutes to 215 minutes and reaction temperatures ranging from 19° C. to 24° C. The five runs were combined, treated with magnesium sulfate (2 kg), allowed to stand for 90 minutes, and filtered through a layer of CELITE filter agent. The filter cake was washed with 1:1 toluene/2-propanol (4 L) and then toluene (16 L), and the filtrate was concentrated under reduced pressure at approximately 40° C. to provide 1.59 kg of N⁴-(2-methylpropyl)[1,5]naphthyridin-3,4-diamine as an oil.

Parts A through D were repeated on the same scale to provide an additional 1.236 kg of N⁴-(2-methylpropyl)[1,5]naphthyridin-3,4-diamine as an oil.

### Part E

Under an atmosphere of argon, diethoxymethyl acetate (2.24 L, 13.7 mol) was added to a solution of N⁴-(2-methylpropyl)[1,5]naphthyridin-3,4-diamine (2.826 kg, 13.07 mol) in toluene (32.5 L) with continuous stirring while maintaining the reaction temperature at or below 30.3° C. The reaction mixture was stirred for 40 minutes at a temperature of 30.1° C. to 30.3° C., heated to reflux over a period of 45 minutes, heated at reflux (92.5° C. to 98.5° C.) for 185 minutes, and allowed to cool to room temperature overnight. Saturated aqueous potassium carbonate (6 L) was added, and the resulting mixture was stirred for 32 minutes at a temperature of 29.3° C. to 30.3° C. and subsequently allowed to stand for 53 minutes. The organic fraction was separated and concentrated under reduced pressure at a temperature of 55° C. to 65° C. over a period of seven hours. The resulting oil was triturated with heptane (3 L) at 20° C. to form a solid, which was isolated by filtration with agitation, washed with cold heptane (1.5 liters (L) at 5° C.) and allowed to air-dry to provide 2.593 kilograms (kg) of 1-(2-methylpropyl)-1H-imidazo[4,5-c][1,5]naphthyridine.

### Part F

A solution of 1-(2-methylpropyl)-1H-imidazo[4,5-c][1,5]naphthyridine (2.593 kg, 11.46 mol) in chloroform (2.8 L) was stirred for one hour at a temperature of 17.5° C. to 18° C., and then 3-chloroperoxybenzoic acid (2.864 g of 70% pure material, 11.6 mol) was added in five portions approximately five minutes apart. During the addition, the temperature of the reaction increased from 16.4° C. to 26.1° C. After the addition, the reaction mixture was stirred for 20 hours, and the reaction temperature decreased from 26.1° C. to 17.5° C. The reaction mixture was then stirred for thirty minutes with 1% w/w aqueous sodium carbonate (4×3.2 L, 30 minutes between washings). The organic fraction was concentrated under reduced pressure at 40° C. The residue (4.6 kg) was triturated with diethyl ether (7.5 L) for 68 minutes, and the resulting solid was isolated by filtration with agitation, washed with diethyl ether (4.5 L), and dried under suction to provide 3.304 kg of 1-(2-methylpropyl)-5-oxido-1H-imidazo[4,5-c][1,5]naphthyridine.

### Part G

Aqueous ammonium hydroxide (28%) was added with continuous stirring to a solution of the material from Part F in dichloromethane (34 L) while maintaining the reaction temperature at or below 11.5° C. With continuous stirring, p-toluenesulfonyl chloride (1.786 kg, 9.368 mol) was added in portions over a period of one hour while maintaining the reaction temperature at 16.4° C. to 25° C. The reaction was stirred for 140 minutes, additional p-toluenesulfonyl chloride (180 g, 0.94 mol) was added, and the reaction was stirred for one additional hour. Water (21 L) was added to the reaction mixture, and the resulting mixture was stirred for 30 minutes and allowed to stand for 14.5 hours. The organic fraction was separated and concentrated under reduced pressure at 40° C. over a period of 8.25 hours. The residue (1.004 kg) was heated at reflux in acetonitrile (10.04 L) for 128 minutes. The suspension was allowed to cool to 20° C., and the resulting solid was isolated by filtration, washed with cold acetonitrile (1.4 L at 4° C.), and air-dried to provide 360 g of 1-(2-methylpropyl)-1H-imidazo[4,5-c][1,5]naphthyridin-4-amine. A solid was present in the reserved aqueous fraction, and the solid was isolated by filtration, washed with water (4×2000 mL), and dried under suction to provide 1.925 kg of 1-(2-methylpropyl)-1H-imidazo[4,5-c][1,5]naphthyridin-4-amine. The two solids were combined and heated at reflux in 90% w/w methanol/water (22.85 L) for 310 minutes, and the suspension was allowed to cool to 24.1° C. overnight. The resulting solid was isolated by filtration, washed with cold 90% w/w methanol/water (1.5 L at 5° C.), and dried in a vacuum oven at 75° C. and 1×10⁵ pascals (Pa) for five days to provide 1.368 kg of 1-(2-methylpropyl)-1H-imidazo[4,5-c][1,5]naphthyridin-4-amine as a light yellow solid.

### Part H

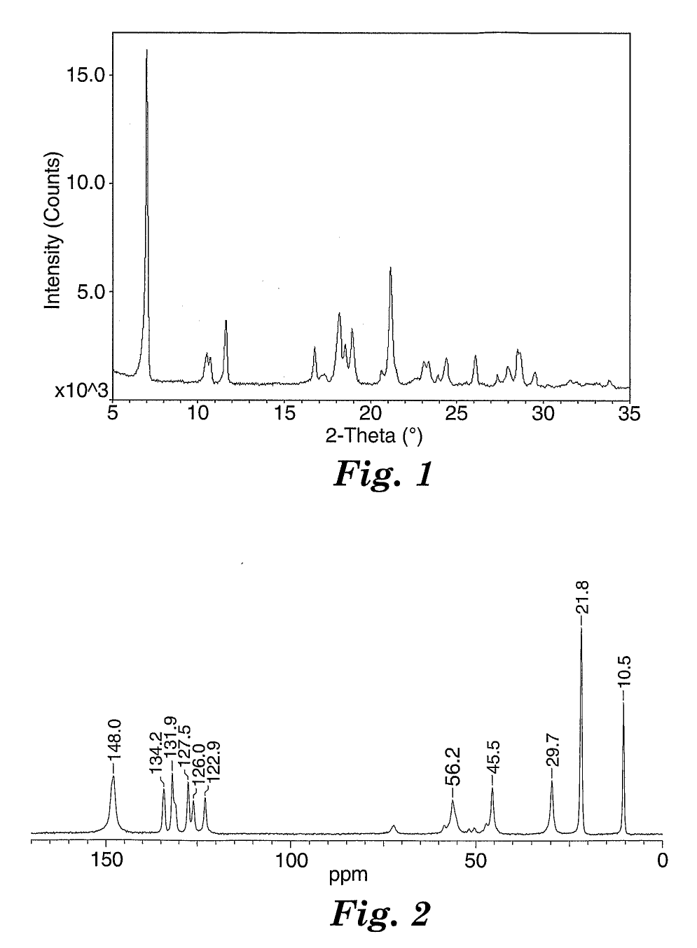

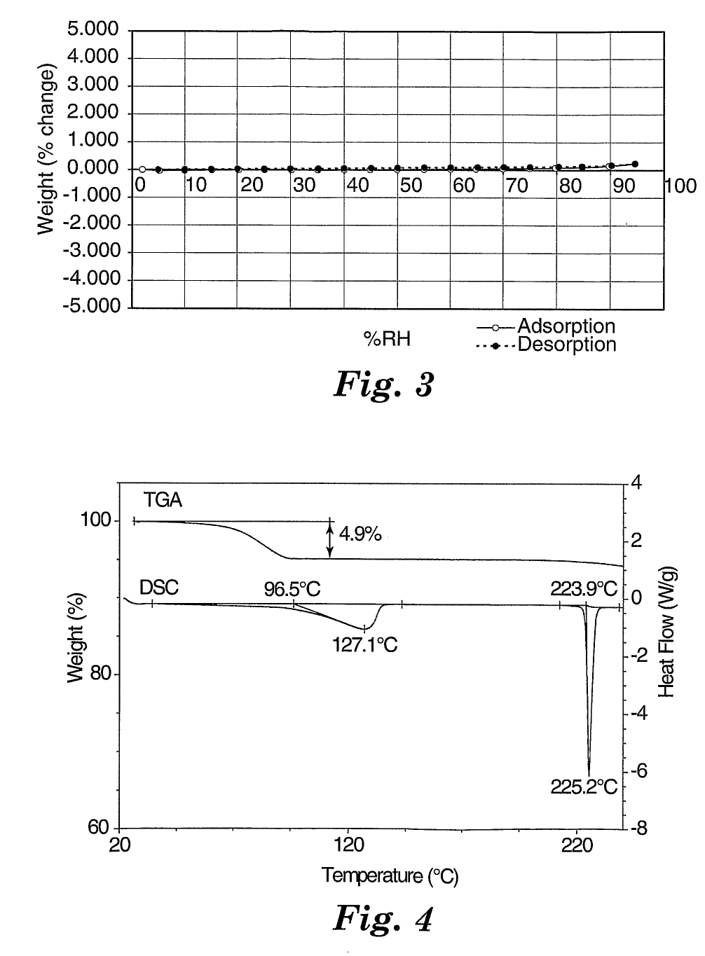

With stirring, 1-(2-methylpropyl)-1H-imidazo[4,5-c][1,5]naphthyridin-4-amine (300 g, 1.24 mol) followed by a rinse of 2% (v/v) water/isopropyl alcohol (100 mL) was added to a vessel containing 2% (v/v) water/isopropyl alcohol (2000 mL), and the resulting mixture was heated to 81° C. A solution of ethanesulfonic acid (151 g of 95%, 1.37 mol) in 2% (v/v) water/isopropyl alcohol (600 mL) was added slowly to the reaction mixture over a period of approximately 20 minutes. During the addition, the mixture became a clear solution, and the temperature was 81° C.-82° C. The addition funnel was rinsed with 2% v/v water/isopropyl alcohol (320 mL); the temperature dropped during the addition but returned to reflux. The resulting solution was heated at reflux for 8 minutes and then cooled slowly to room temperature at a rate of 0.2° C./minute (57° C. over 234 minutes). The resulting slurry was then further cooled at a rate of 0.2° C./minute to a temperature of 0° C. to 5° C. (i.e., the slurry was cooled 21° C. over a period of 130 minutes). The solid was isolated by filtration using cold 2% (v/v) water/isopropyl alcohol (400 mL, 3.5° C.) to rinse and aid in the transfer. The solid was washed with cold 2% (v/v) water/isopropyl alcohol (300 mL, 3.5° C.), dried at about 43° C. under 1.69×10³ Pa-1.70×10³ Pa (169 mbar-170 mbar) for 23 hours and 40 minutes to provide 455 g of 1-(2-methylpropyl)-1H-imidazo[4,5-c][1,5]naphthyridin-4-amine ethanesulfonate monohydrate. The material was processed in a Hamilton Beach 14-speed blender to provide a white “cotton-like” solid, mp 221.5° C.-223.4° C. Anal. Calcd. for C₁₃H₁₅N₅.C₂H₆O₃S.H₂O: C, 48.77; H, 6.28; N, 18.96. Found: C, 48.70; H, 6.25; N, 18.96. This material was characterized by powder X-ray diffraction analysis, ¹³C NMR spectroscopy, water sorption analysis, thermogravimetric analysis, and differential scanning calorimetry. The powder X-ray diffraction pattern and ¹³C NMR spectra are shown in FIGS. 1 and 2. The DSC/TGA overlay is shown in FIG. 3, and the water sorption isotherm is shown in FIG. 4.

### Test Method

In the examples below the serum and intravaginal cytokine data were obtained using the following general test method.

Rats were acclimated to collars (Lomir Biomedical, Malone, N.Y.) around the neck on two consecutive days prior to actual dosing. Rats were collared to prevent ingestion of the drug. Animals were then dosed intravaginally with 50 μL of gel. Single dosed rats received one intravaginal dose with samples collected at various times following dosing. Multiple dosed rats were dosed as described in the examples below with samples collected at various times following the final dose. Blood was collected by cardiac puncture. Blood was allowed to clot briefly at room temperature and serum was separated from the clot via centrifugation. The serum was stored at −20° C. until it was analyzed for cytokine concentrations.

Following blood collection, the rats were euthanized and their vaginal tract, including the cervix, was then removed and the tissue was weighed, placed in a sealed 1.8 mL cryovial and flash frozen in liquid nitrogen. The frozen vaginal tissue sample was then suspended in 1.0 mL of RPMI medium (Celox, St. Paul, Minn.) containing 10% fetal bovine serum (Atlas, Fort Collins, Colo.), 2 mM L-glutamine, penicillin/streptomycin and 2-mercaptoethanol (RPMI complete) combined with a protease inhibitor cocktail set III (Calbiochem, San Diego, Calif.). The tissue was homogenized using a Tissue Tearor (Biospec Products, Bartlesville, Okla.) for approximately one minute. The tissue suspension was then centrifuged at 2000 rpm for 10 minutes under refrigeration to pellet the debris, and the supernatant collected and stored at −20° C. until analyzed for cytokine concentrations.

ELISA kits for rat tumor necrosis factor-alpha (TNF) were purchased from BD PharMingen (San Diego, Calif.) and the rat monocyte chemoattractant protein-1 (MCP-1) ELISA kits were purchased from BioSource Intl. (Camarillo, Calif.). Both kits were performed according to manufacturer's specifications. Results for both TNF and MCP-1 are expressed in pg/mL and are normalized per 200 mg of tissue. The sensitivity of the TNF ELISA, based on the lowest value used to form the standard curve, is 63 pg/mL and for the MCP-1 ELISA it is 12 pg/mL.

### Examples 1-5

The gels shown in Table 1 below were prepared using the following method.
Step 1: The parabens were dissolved in either propylene glycol or hexylene glycol. An aqueous solution of ethanesulfonic acid was added to the glycol solution. 1-(2-Methylpropyl)-1H-imidazo[4,5-c][1,5]naphthyridin-4-amine (drug) was then dissolved in the solution.
Step 2: Edetate disodium was dissolved in water. Carbomer 974P and xanthan gum, if used, were dispersed in the solution.
Step 3: The solution from Step 1 was added to the dispersion from Step 2 while mixing. 20% Tromethamine solution was added to the mixture to bring the final formulation pH to 4.
   The gels were filled into alloy aluminum tubes.

TABLE 1

| | Gels (% w/w) | | | | |
|---|---|---|---|---|---|
| Ingredient | Ex 1 | Ex 2 | Ex 3 | Ex 4 | Ex 5 |
| Drug | 0.01 | 0.03 | 0.10 | 0.30 | 1.00 |
| 1.0% Ethanesulfonic | 0.46 | 1.37 | 4.56 | 13.69 | 0.00 |
| acid solution | | | | | |
| 5.0% Ethanesulfonic | 0.00 | 0.00 | 0.00 | 0.00 | 9.13 |
| acid solution | | | | | |
| Carbomer 974P | 1.50 | 1.50 | 1.70 | 2.00 | 1.00 |
| Xanthan gum | 0.00 | 0.00 | 0.00 | 0.00 | 1.00 |
| Propylene glycol | 15.00 | 15.00 | 15.00 | 30.00 | 0.00 |
| Hexylene glycol | 0.00 | 0.00 | 0.00 | 0.00 | 30.00 |
| Methylparaben | 0.15 | 0.15 | 0.15 | 0.15 | 0.15 |
| Propylparaben | 0.03 | 0.03 | 0.03 | 0.03 | 0.03 |
| Edetate disodium | 0.05 | 0.05 | 0.05 | 0.05 | 0.05 |
| 20% Tromethamine | 0.54 | 0.85 | 1.25 | 1.50 | 1.80 |
| solution | | | | | |
| Purified water | 82.26 | 81.02 | 77.16 | 52.28 | 55.84 |
| pH | 3.8 | 4.0 | 4.0 | 4.1 | 3.2 |
| Viscosity (cps) | 6000 | 7000 | 7100 | 3400 | 3300 |

The ability of the gel of Example 3 to induce cytokines following a single dose was determined using the test method described above. The results are shown in Table 2 below where each value is the mean of 5 animals±SEM (standard error of the mean).

TABLE 2

| Time | | Cytokine Concentrations | | | |
|---|---|---|---|---|---|
| (hours) | | TNF (pg/mL) | | MCP-1 (pg/mL) | |
| Post Dose | Gel | Serum | Tissue | Serum | Tissue |
| 0 | None | <63 | 126 ± 13 | 73 ± 9 | 33 ± 2 |
| 4 | ¹Vehicle | <63 | 108 ± 7 | 130 ± 32 | 35 ± 2 |
| 1 | Example 3 | <63 | 401 ± 104 | 124 ± 22 | 52 ± 17 |
| 2 | Example 3 | <63 | 862 ± 160 | 104 ± 17 | 474 ± 113 |
| 4 | Example 3 | <63 | 741 ± 133 | 75 ± 26 | 761 ± 211 |
| 6 | Example 3 | <63 | 298 ± 42 | 172 ± 17 | 1065 ± 131 |
| 8 | Example 3 | <63 | 201 ± 35 | 196 ± 24 | 832 ± 187 |
| 16 | Example 3 | <63 | 153 ± 15 | 71 ± 24 | 230 ± 91 |
| 24 | Example 3 | <63 | 138 ± 6 | 189 ± 24 | 324 ± 122 |

¹Vehicle (2.00% carbomer 974, 30.00% propylene glycol, 0.15% methylparaben, 0.03% propylparaben, 0.05% edetate sodium, 0.58% 20% tromethamine solution, and 67.19% water)

The ability of the gels of Examples 3 and 5 to induce cytokines following both single and multiple dosing was determined using the test method described above. The multiple dosed animals received an intravaginal dose once a day, 2 times a week (Monday and Thursday) over 3 weeks for a total of 6 doses. The results are shown in Table 3 below where each value is the mean of 5 animals±SEM.

TABLE 3

| Time | | | | | | |
|---|---|---|---|---|---|---|
| (hours) | | | Cytokine Concentrations | | | |
| Post | | | TNF (pg/mL) | | MCP-1 (pg/mL) | |
| Dose | Gel | Dosing | Serum | Tissue | Serum | Tissue |
| 0 | None | | <63 | 222 ± 72 | 89 ± 7 | 39 ± 12 |
| 4 | ¹Vehicle | Single | <63 | 159 ± 38 | 76 ± 14 | 38 ± 7 |
| 2 | Example 3 | Single | <63 | 693 ± 105 | 41 ± 12 | 323 ± 76 |
| 4 | Example 3 | Single | <63 | 296 ± 110 | 96 ± 12 | 252 ± 51 |
| 6 | Example 3 | Single | <63 | 314 ± 42 | 95 ± 9 | 718 ± 122 |
| 4 | ²Vehicle | Single | <63 | 261 ± 35 | 105 ± 14 | 176 ± 30 |
| 2 | Example 5 | Single | 271 ± 71 | 665 ± 101 | 345 ± 105 | 1188 ± 359 |
| 4 | Example 5 | Single | <63 | 245 ± 48 | 614 ± 101 | 869 ± 86 |
| 6 | Example 5 | Single | <63 | 216 ± 15 | 418 ± 142 | 778 ± 190 |
| 4 | ¹Vehicle | Multiple | <63 | 43 ± 3 | 45 ± 12 | 25 ± 4 |
| 2 | Example 3 | Multiple | <63 | 564 ± 6 | 66 ± 13 | 341 ± 46 |
| 4 | Example 3 | Multiple | <63 | 1078 ± 333 | 48 ± 13 | 970 ± 264 |
| 6 | Example 3 | Multiple | <63 | 181 ± 72 | 85 ± 12 | 1196 ± 384 |
| 4 | ²Vehicle | Multiple | <63 | 54 ± 14 | 65 ± 9 | 77 ± 31 |
| 2 | Example 5 | Multiple | <63 | 646 ± 221 | 125 ± 42 | 902 ± 259 |
| 4 | Example 5 | Multiple | <63 | 601 ± 286 | 93 ± 31 | 1194 ± 398 |
| 6 | Example 5 | Multiple | <63 | 174 ± 44 | 174 ± 47 | 1414 ± 391 |

¹Vehicle (2.00% carbomer 974, 30.00% propylene glycol, 0.15% methylparaben, 0.03% propylparaben, 0.05% edetate sodium, 0.58% 20% tromethamine solution, and 67.19% water)
¹Vehicle (1.00% carbomer 974, 1.00% xanthan gum, 30.00% hexylene glycol, 0.15% methylparaben, 0.03% propylparaben, 0.05% edetate sodium, and 67.19% water)

### Examples 6-8

The gels shown in Table 4 below were prepared using the following method.
Step 1: The parabens were dissolved in propylene glycol. 1-(2-Methylpropyl)-1H-imidazo[4,5-c][1,5]naphthyridin-4-amine ethanesulfonate monohydrate was then dissolved in the solution.
Step 2: Edetate disodium was dissolved in water. Carbomer 974P was dispersed in the solution.
Step 3: The solution from Step 1 was added to the dispersion from Step 2 while mixing. 20% Tromethamine solution was added to the mixture to adjust the pH.

TABLE 4

| | Gels (% w/w) | | |
|---|---|---|---|
| Ingredient | Ex 6 | Ex 7 | Ex 8 |
| Drug (ethanesulfonate monohydrate) | 0.0153 | 0.0469 | 0.153 |
| Carbomer 974P | 1.50 | 1.50 | 1.70 |
| Propylene glycol | 15.00 | 15.00 | 15.00 |
| Methylparaben | 0.15 | 0.15 | 0.15 |
| Propylparaben | 0.03 | 0.03 | 0.03 |
| Edetate disodium | 0.05 | 0.05 | 0.05 |
| 20% Tromethamine solution | 0.54 | 0.85 | 1.25 |
| Purified water | 82.7147 | 82.5231 | 81.667 |
| pH | 3.8 | 4.0 | 4.0 |
| Viscosity (cps) | 6000 | 7000 | 7000 |

### Examples 9 and 10

The gels shown in Table 5 below were prepared using the general method of Examples 6-8.

TABLE 5

Gels (% w/w)

| Ingredient | Ex 9 | Ex 10 |
|---|---|---|
| Drug (ethanesulfonate monohydrate) | 0.0015 | 0.0046 |
| Carbomer 974P | 1.5 | 1.5 |
| Propylene glycol | 15 | 15 |
| Methylparaben | 0.15 | 0.15 |
| Propylparaben | 0.03 | 0.03 |
| Edetate disodium | 0.05 | 0.05 |
| 20% Tromethamine solution | 0.5 | 0.5 |
| Purified water | 82.7685 | 82.7654 |
| pH | 4.0 | 4.0 |

The ability of the gels of Examples 1, 3, 9, and 10 to induce cytokines following a single dose was determined using the test method described above. The results are shown in Table 6 below where each value is the mean of 5 animals±SEM.

TABLE 6

| Time | | Cytokine Concentrations | | | |
|---|---|---|---|---|---|
| (hours) | | TNF (pg/mL) | | MCP-1 (pg/mL) | |
| Post Dose | Gel | Serum | Tissue | Serum | Tissue |
| 0 | None | <63 | 210 ± 20 | 122 ± 12 | 21 ± 4 |
| 4 | ¹Vehicle | <63 | 118 ± 19 | 120 ± 15 | 12 ± 1 |
| 2 | Example 9 | <63 | 172 ± 55 | 76 ± 14 | 21 ± 10 |
| 4 | Example 9 | <63 | 95 ± 13 | 119 ± 13 | 14 ± 2 |
| 8 | Example 9 | <63 | 151 ± 14 | 94 ± 21 | 17 ± 3 |
| 2 | Example 10 | <63 | 221 ± 58 | 74 ± 16 | 34 ± 15 |
| 4 | Example 10 | <63 | 149 ± 19 | 96 ± 12 | 90 ± 18 |
| 8 | Example 10 | <63 | 132 ± 22 | 126 ± 16 | 46 ± 19 |
| 2 | Example 1 | <63 | 426 ± 139 | 25 ± 6 | 231 ± 67 |
| 4 | Example 1 | <63 | 243 ± 35 | 88 ± 23 | 236 ± 66 |
| 8 | Example 1 | <63 | 157 ± 30 | 162 ± 16 | 134 ± 49 |
| 2 | Example 3 | <63 | 702 ± 285 | 81 ± 15 | 306 ± 154 |
| 4 | Example 3 | <63 | 1074 ± 154 | 135 ± 18 | 981 ± 103 |
| 8 | Example 3 | <63 | 271 ± 38 | 175 ± 33 | 895 ± 344 |

¹Vehicle (2.00% carbomer 974, 30.00% propylene glycol, 0.15% methylparaben, 0.03% propylparaben, 0.05% edetate sodium, 0.58% 20% tromethamine solution, and 67.19% water)

### Examples 11-18

The gels of Examples 11, 12, and 14 were prepared using the general method of Examples 6-8. The gels of Examples 13, 15, 16, 17, and 18 were prepared using the following method.
Step 1: The parabens were dissolved in propylene glycol (approximately 66 wt % of the total amount used to achieve the final wt % in the gel). 1-(2-Methylpropyl)-1H-imidazo[4,5-c][1,5]naphthyridin-4-amine ethanesulfonate monohydrate was then dissolved in the solution.
Step 2: Edetate disodium was dissolved in water. The remainder of the propylene glycol was added. Carbomer 974P was added and mixing was continued until the carbomer was completely hydrated.
Step 3: The solution from Step 1 was added to the dispersion from Step 2 while mixing. After mixing was complete the pH was measured.

TABLE 7

| | Gels (% w/w) | | | | | | | |
|---|---|---|---|---|---|---|---|---|
| Ingredient | Ex 11 | Ex 12 | Ex 13 | Ex 14 | Ex 15 | Ex 16 | Ex 17 | Ex 18 |
| Drug | 0.153 | 0.153 | 0.153 | 0.459 | 0.459 | 0.765 | 1.531 | 4.593 |
| (ethanesulfonate | | | | | | | | |
| monohydrate) | | | | | | | | |
| Carbomer 974P | 2.10 | 2.10 | 2.10 | 2.00 | 2.10 | 2.10 | 2.50 | 2.30 |
| Propylene glycol | 15.00 | 30.00 | 60.00 | 30.00 | 60.00 | 60.00 | 60.00 | 60.00 |
| Methylparaben | 0.15 | 0.15 | 0.15 | 0.15 | 0.15 | 0.15 | 0.15 | 0.15 |
| Propylparaben | 0.03 | 0.03 | 0.03 | 0.03 | 0.03 | 0.03 | 0.03 | 0.03 |
| Edetate disodium | 0.05 | 0.05 | 0.05 | 0.05 | 0.05 | 0.05 | 0.05 | 0.05 |
| Tromethamine | 0.27 | 0.27 | 0.00 | 0.35 | 0.00 | 0.00 | 0.00 | 0.00 |
| Purified water | 82.25 | 67.25 | 37.52 | 66.96 | 37.21 | 36.91 | 35.74 | 32.88 |
| pH | 4.0 | 4.2 | 3.8 | 4.3 | 3.6 | 3.2 | 2.9 | 2.5 |

The ability of the gels of Examples 11-18 to induce cytokines following a single dose was determined using the test method described above with the following exceptions: the tissue samples were centrifuged at 3000 rpm for 10 minutes, all samples were subject to a dilution factor of 1:2 for TNF and 1:4 for MCP-1, and the sensitivity of the TNF ELISA, based on the lowest value used to form the standard curve, was 31 pg/mL. The results are shown in Table 8 below where each value is the mean of 3 animals±SEM.

TABLE 8

| Time | | Cytokine Concentrations | | | |
|---|---|---|---|---|---|
| (hours) | | TNF (pg/mL) | | MCP-1 (pg/mL) | |
| Post Dose | Gel | Serum | Tissue | Serum | Tissue |
| 0 | None | ND | 223 ± 24 | ND | 36 ± 7 |
| 0 | ¹Example 11 | 101 ± 7 | ND | 164 ± 21 | ND |
| 4 | Example 11 | 90 ± 4 | 490 ± 39 | 299 ± 60 | 642 ± 326 |
| 6 | Example 11 | 83 ± 8 | 333 ± 10 | 342 ± 41 | 853 ± 49 |
| 4 | Example 12 | 67 ± 1 | 1074 ± 289 | 323 ± 40 | 1015 ± 257 |
| 6 | Example 12 | 81 ± 8 | 292 ± 5 | 362 ± 35 | 316 ± 37 |
| 4 | Example 13 | 67 ± 36 | 834 ± 73 | 271 ± 20 | 1222 ± 374 |
| 6 | Example 13 | 62 ± 5 | 150 ± 6 | 293 ± 43 | 215 ± 64 |
| 4 | Example 14 | 85 ± 15 | 853 ± 42 | 232 ± 37 | 874 ± 232 |
| 6 | Example 14 | 68 ± 5 | 183 ± 35 | 305 ± 29 | 223 ± 33 |
| 4 | Example 15 | 83 ± 24 | 1468 ± 618 | 170 ± 20 | 1086 ± 59 |
| 6 | Example 15 | 51 ± 6 | 207 ± 39 | 246 ± 14 | 212 ± 24 |
| 4 | Example 16 | 86 ± 19 | 446 ± 144 | 444 ± 193 | 646 ± 114 |
| 6 | Example 16 | 80 ± 25 | 245 ± 67 | 257 ± 27 | 368 ± 112 |
| 4 | Example 17 | 77 ± 3 | 838 ± 254 | 746 ± 203 | 831 ± 230 |
| 6 | Example 17 | 95 ± 9 | 312 ± 70 | 580 ± 301 | 396 ± 119 |
| 4 | Example 18 | 118 ± 43 | 378 ± 104 | 1248 ± 496 | 1133 ± 71 |
| 6 | Example 18 | 100 ± 14 | 251 ± 52 | 1072 ± 397 | 1003 ± 284 |

¹Blood samples taken prior to dosing
ND = not determined

The ability of the gels of Examples 15, 17, and 18 to induce cytokines following a single dose was determined using the test method described above. The results are shown in Table 9 below where each value is the mean of 6 animals±SEM.

TABLE 9

| Time | | Cytokine Concentrations | | | |
|---|---|---|---|---|---|
| (hours) | | TNF (pg/mL) | | MCP-1 (pg/mL) | |
| Post Dose | Gel | Serum | Tissue | Serum | Tissue |
| 0 | None | 0 ± 0 | 382 ± 215 | 207 ± 32 | 6 ± 6 |
| 2 | ¹Vehicle | 54 ± 54 | 240 ± 59 | 174 ± 36 | 32 ± 12 |
| 1 | Example 15 | 0 ± 0 | 930 ± 241 | 129 ± 21 | 72 ± 17 |
| 2 | Example 15 | 0 ± 0 | 892 ± 193 | 174 ± 25 | 315 ± 78 |
| 4 | Example 15 | 0 ± 0 | 833 ± 270 | 230 ± 40 | 651 ± 150 |
| 1 | Example 17 | 0 ± 0 | 695 ± 165 | 120 ± 11 | 63 ± 17 |
| 2 | Example 17 | 134 ± 134 | 1202 ± 201 | 626 ± 360 | 459 ± 88 |
| 4 | Example 17 | 18 ± 18 | 1841 ± 421 | 657 ± 420 | 852 ± 275 |
| 1 | Example 18 | 0 ± 0 | 1170 ± 110 | 150 ± 34 | 245 ± 63 |
| 2 | Example 18 | 103 ± 79 | 1020 ± 158 | 547 ± 353 | 551 ± 74 |
| 4 | Example 18 | 21 ± 21 | 1007 ± 306 | 1074 ± 487 | 1122 ± 298 |

¹Vehicle (2.50% carbomer 974, 60.00% propylene glycol, 0.15% methylparaben, 0.03% propylparaben, 0.05% edetate sodium, and 37.27% water)

### Examples 19-23

The gels shown in Table 10 below were prepared using the following method.
Step 1: The parabens were dissolved in propylene glycol (approximately 66 wt % of the total amount used to achieve the final wt % in the gel). 1-(2-Methylpropyl)-1H-imidazo[4,5-c][1,5]naphthyridin-4-amine ethanesulfonate monohydrate was then dissolved in the solution.
Step 2: Edetate disodium was dissolved in water. The remainder of the propylene glycol was added. Carbomer 974P and xanthan gum, if used, were added sequentially and mixing was continued until the thickener(s) was completely hydrated.
Step 3: The solution from Step 1 was added to the dispersion from Step 2 while mixing. After mixing was complete the pH was measured.

TABLE 10

| | Gels (% w/w) | | | | |
|---|---|---|---|---|---|
| Ingredient | Ex 19 | Ex 20 | Ex 21 | Ex 22 | Ex 23 |
| Drug (ethanesulfonate | 1.531 | 1.531 | 1.531 | 1.531 | 1.531 |
| monohydrate) | | | | | |
| Carbomer 974P | 1.25 | 1.50 | 1.50 | 2.00 | 2.50 |
| Xanthan gum | 1.75 | 1.50 | 1.50 | 1.00 | 0.00 |
| Propylene glycol | 50.00 | 50.00 | 60.00 | 60.00 | 60.00 |
| Methylparaben | 0.15 | 0.15 | 0.15 | 0.15 | 0.15 |
| Propylparaben | 0.03 | 0.03 | 0.03 | 0.03 | 0.03 |
| Edetate disodium | 0.05 | 0.05 | 0.05 | 0.05 | 0.05 |
| Purified water | 45.24 | 45.24 | 35.24 | 35.24 | 35.74 |
| pH | 3.0 | 2.8 | 3.2 | 3.1 | 2.9 |

The ability of the gels of Examples 19-23 to induce cytokines following a single dose was determined using the test method described above. The results are shown in Table 11 below where each value is the mean of 4 animals±SEM.

TABLE 11

| Time | | Cytokine Concentrations | | | |
|---|---|---|---|---|---|
| (hours) | | TNF (pg/mL) | | MCP-1 (pg/mL) | |
| Post Dose | Gel | Serum | Tissue | Serum | Tissue |
| 4 | None | 133 ± 15 | 185 ± 22 | 152 ± 20 | 81 ± 10 |
| 4 | Example 19 | 86 ± 2 | 1043 ± 479 | 359 ± 158 | 1663 ± 217 |
| 4 | Example 20 | 109 ± 13 | 701 ± 247 | 331 ± 70 | 1834 ± 315 |
| 4 | Example 21 | 103 ± 17 | 831 ± 130 | 177 ± 18 | 683 ± 91 |
| 4 | Example 22 | 126 ± 12 | 516 ± 81 | 526 ± 173 | 1539 ± 469 |
| 4 | Example 23 | 99 ± 25 | 1300 ± 593 | 575 ± 406 | 571 ± 272 |

### Examples 24-27

The gels of Examples 24-26 were prepared using the general method of Examples 19-23. The gel of Example 27 was prepared using the general method of Examples 6-8 except that the tromethamine was omitted.

TABLE 12

| | Gels (% w/w) | | | |
|---|---|---|---|---|
| Ingredient | Ex 24 | Ex 25 | Ex 26 | Ex 27 |
| Drug (ethanesulfonate | 1.531 | 1.531 | 1.531 | 1.531 |
| monohydrate) | | | | |
| Carbomer 974P | 2.00 | 1.00 | 2.00 | 2.50 |
| Xanthan gum | 1.00 | 2.00 | 1.00 | 0.00 |
| Propylene glycol | 50.00 | 50.00 | 60.00 | 60.00 |
| Methylparaben | 0.15 | 0.15 | 0.15 | 0.15 |
| Propylparaben | 0.03 | 0.03 | 0.03 | 0.03 |
| Edetate disodium | 0.05 | 0.05 | 0.05 | 0.05 |
| 20% Tromethamine solution | 1.44 | 1.00 | 0.59 | 0.00 |
| Purified water | 43.80 | 44.24 | 34.65 | 35.74 |
| pH | 3.4 | 3.5 | 3.4 | 3.1 |
| Viscosity (cps) | 11000 | 7550 | ND | ND |

ND = not determined

The ability of the gels of Examples 24-27 to induce cytokines following a single dose was determined using the test method described above. The results are shown in Table 13 below where each value is the mean of 6 animals±SEM.

TABLE 13

| Time | | Cytokine Concentrations | | | |
|---|---|---|---|---|---|
| (hours) | | TNF (pg/mL) | | MCP-1 (pg/mL) | |
| Post Dose | Gel | Serum | Tissue | Serum | Tissue |
| 0 | None | 62 ± 46 | 317 ± 87 | 230 ± 21 | 26 ± 4 |
| 2 | Example 24 | 10 ± 10 | 571 ± 81 | 168 ± 26 | 370 ± 89 |
| 4 | Example 24 | 51 ± 32 | 938 ± 133 | 240 ± 52 | 1133 ± 199 |
| 2 | Example 25 | 66 ± 35 | 558 ± 61 | 191 ± 39 | 449 ± 107 |
| 4 | Example 25 | 75 ± 21 | 661 ± 101 | 185 ± 21 | 1058 ± 254 |
| 2 | Example 26 | 161 ± 39 | 699 ± 121 | 178 ± 32 | 401 ± 107 |
| 4 | Example 26 | 15 ± 15 | 569 ± 90 | 277 ± 72 | 1215 ± 260 |
| 2 | Example 27 | 69 ± 31 | 804 ± 240 | 143 ± 31 | 330 ± 125 |
| 4 | Example 27 | 90 ± 74 | 912 ± 244 | 424 ± 203 | 1089 ± 333 |

### Examples 28-32

The gels of Examples 28-32 were prepared using the general method of Examples 6-8 except that both the carbomer and xanthan gum were added in step 2.

TABLE 14

| | Gels (% w/w) | | | | |
|---|---|---|---|---|---|
| Ingredient | Ex 28 | Ex 29 | Ex 30 | Ex 31 | Ex 32 |
| Drug (ethanesulfonate monohydrate) | 0.15 | 0.45 | 0.75 | 1.50 | 4.50 |
| Carbomer 974P | 2.20 | 2.33 | 2.50 | 2.00 | 1.75 |
| Xanthan gum | 1.10 | 1.17 | 1.25 | 1.00 | 0.88 |
| Propylene glycol | 15.00 | 30.00 | 30.00 | 40.00 | 60.00 |
| Methylparaben | 0.15 | 0.15 | 0.15 | 0.15 | 0.15 |
| Propylparaben | 0.03 | 0.03 | 0.03 | 0.03 | 0.03 |
| Edetate disodium | 0.05 | 0.05 | 0.05 | 0.05 | 0.05 |
| 20% Tromethamine solution | 0.50 | 1.25 | 0.67 | 1.70 | 2.00 |
| Purified water | 80.82 | 64.57 | 64.60 | 53.57 | 30.64 |
| pH | 4.0 | 4.0 | 3.5 | 3.5 | 3.0 |
| Viscosity | 8900 | 11000 | 8600 | 12000 | 17000 |

### Example 33

The gel shown in Table 15 below was prepared using the following method.
Step 1: Edetate disodium was dissolved in water (approximately 99% of the total amount used to achieve the final wt % in the gel).
Step 2: The parabens were dissolved in propylene glycol. 1-(2-Methylpropyl)-1H-imidazo[4,5-c][1,5]naphthyridin-4-amine ethanesulfonate monohydrate was then dissolved in the solution.
Step 3: Carbomer 974P and xanthan gum were added sequentially to the solution from Step 1 while mixing and mixing was continued until the thickeners were completely hydrated.
Step 4: The solution from step 2 was added to the dispersion from step 3 while mixing.
Step 5: Tromethamine was dissolved in water (20% by weight tromethamine) and the solution was added to the gel from step 4 while mixing. Mixing was continued until the is gel was uniform. After mixing was complete the pH was measured.

TABLE 15

| Ingredient | Example 33 Gel (% w/w) |
|---|---|
| Drug (ethanesulfonate monohydrate) | 0.15 |
| Carbomer 974P | 2.20 |
| Xanthan gum | 1.10 |
| Propylene glycol | 15.00 |
| Methylparaben | 0.15 |
| Propylparaben | 0.03 |
| Edetate disodium | 0.05 |
| Tromethamine | 0.20 |
| Purified water | 81.12 |
| pH | 3.8 |
| Viscosity (cps) | 10700 |

### Example 34

The gel shown in Table 16 below was prepared using the following method.
Step 1: The parabens were dissolved in propylene glycol. 1-(2-Methylpropyl)-1H-imidazo[4,5-c][1,5]naphthyridin-4-amine ethanesulfonate monohydrate was then dissolved in the solution.
Step 2: Edetate disodium was dissolved in water (approximately half of the total amount used to achieve the final wt % in the gel).
Step 3: Carbomer 974P, xanthan gum, the solution from step 2, and water (approximately half of the total amount used to achieve the final wt % in the gel) were added sequentially to the solution from Step 1 while mixing and mixing was continued until the thickeners were completely hydrated.
Step 4: Tromethamine was dissolved in water (20% by weight tromethamine) and the solution was added to the gel from step 3 while mixing. Mixing was continued until the gel was uniform. After mixing was complete the pH was measured.

TABLE 16

| Ingredient | Example 34 Gel (% w/w) |
|---|---|
| Drug (ethanesulfonate monohydrate) | 1.50 |
| Carbomer 974P | 2.00 |
| Xanthan gum | 1.00 |
| Propylene glycol | 40.00 |
| Methylparaben | 0.15 |
| Propylparaben | 0.03 |
| Edetate disodium | 0.05 |
| Tromethamine | 0.40 |
| Purified water | 54.87 |
| pH | 3.6 |
| Viscosity (cps) | 7800 |

### Example 35

The gel shown in Table 17 below was prepared using the following method.
Step 1: The parabens were dissolved in propylene glycol. 1-(2-Methylpropyl)-1H-imidazo[4,5-c][1,5]naphthyridin-4-amine ethanesulfonate monohydrate was added in 3 separate portions while mixing. Mixing was continued until the drug was completely dissolved.
Step 2: Edetate disodium was dissolved in water (approximately a third of the total amount used to achieve the final wt % in the gel).
Step 3: Carbomer 974P, xanthan gum, the solution from step 2, and water (approximately two thirds of the total amount used to achieve the final wt % in the gel) were added sequentially to the solution from Step 1 while mixing and mixing was continued until the thickeners were completely hydrated.
Step 4: Tromethamine was dissolved in water (20% by weight tromethamine) and the solution was added to the gel from step 3 while mixing. Mixing was continued until the gel was uniform. After mixing was complete the pH was measured.

TABLE 17

| Ingredient | Example 35 Gel (% w/w) |
|---|---|
| Drug (ethanesulfonate monohydrate) | 4.50 |
| Carbomer 974P | 1.75 |
| Xanthan gum | 0.88 |
| Propylene glycol | 50.00 |
| Methylparaben | 0.15 |
| Propylparaben | 0.03 |
| Edetate disodium | 0.05 |
| Tromethamine | 0.65 |
| Purified water | 41.99 |
| pH | 3.0 |
| Viscosity (cps) | 7100 |

The ability of the gels of Examples 34 and 35 to induce cytokines following a single dose was determined using the test method described above. The results are shown in Table 18 below where each value is the mean of 6 animals±SEM.

TABLE 18

| Time | | Cytokine Concentrations | | | |
|---|---|---|---|---|---|
| (hours) | | TNF (pg/mL) | | MCP-1 (pg/mL) | |
| Post Dose | Gel | Serum | Tissue | Serum | Tissue |
| 1 | Example 34 | 138 ± 55 | 930 ± 303 | 219 ± 33 | 45 ± 22 |
| 2 | Example 34 | 261 ± 160 | 1686 ± 456 | 153 ± 34 | 571 ± 149 |
| 4 | Example 34 | 55 ± 17 | 1515 ± 170 | 87 ± 38 | 1153 ± 159 |
| 1 | Example 35 | 390 ± 180 | 891 ± 78 | 246 ± 29 | 94 ± 25 |
| 2 | Example 35 | 218 ± 61 | 1325 ± 140 | 530 ± 265 | 808 ± 206 |
| 4 | Example 35 | 222 ± 117 | 954 ± 145 | 375 ± 173 | 1277 ± 265 |
| 4 | ¹Vehicle | 83 ± 36 | 513 ± 98 | 212 ± 41 | 90 ± 33 |

¹Vehicle (1.75% carbomer 974, 0.88% xanthan gum, 50.00% propylene glycol, 0.15% methylparaben, 0.03% propylparaben, 0.05% edetate sodium, 0.05 tromethamine, and 47.09% water)

### Example 36

The gel shown in Table 19 below was prepared using the method of Example 35 except that 1-(2-methylpropyl)-1H-imidazo[4,5-c][1,5]naphthyridin-4-amine ethanesulfonate monohydrate was added in 2 separate portions.

TABLE 19

| Ingredient | Example 36 Gel (% w/w) |
|---|---|
| Drug (ethanesulfonate monohydrate) | 3.00 |
| Carbomer 974P | 1.83 |
| Xanthan gum | 0.92 |
| Propylene glycol | 45.00 |
| Methylparaben | 0.15 |
| Propylparaben | 0.03 |
| Edetate disodium | 0.05 |
| Tromethamine | 0.71 |
| Purified water | 48.31 |
| pH | 3.4 |
| Viscosity (cps) | 8500 |

The ability of the gel of Example 36 to induce cytokines following a single dose was determined using the test method described above. The results are shown in Table 20 below where each value is the mean of 6 animals±SEM.

TABLE 20

| Time | | Cytokine Concentrations | | | |
|---|---|---|---|---|---|
| (hours) | | TNF (pg/mL) | | MCP-1 (pg/mL) | |
| Post Dose | Gel | Serum | Tissue | Serum | Tissue |
| 1 | Example 36 | 768 ± 237 | 1826 ± 345 | 154 ± 30 | 145 ± 51 |
| 2 | Example 36 | 684 ± 391 | 2163 ± 121 | 593 ± 349 | 1437 ± 225 |
| 4 | Example 36 | 827 ± 423 | 1334 ± 241 | 904 ± 406 | 1593 ± 333 |
| 4 | ¹Vehicle | 299 ± 478 | 1385 ± 221 | 208 ± 46 | 107 ± 32 |

¹Vehicle (1.75% carbomer 974, 0.88% xanthan gum, 50.00% propylene glycol, 0.15% methylparaben, 0.03% propylparaben, 0.05% edetate sodium, 0.05 tromethamine, and 47.09% water)

### Example 37

The gel shown in Table 21 below was prepared using the following method.
Step 1: The parabens were dissolved in propylene glycol. 1-(2-Methylpropyl)-1H-imidazo[4,5-c][1,5]naphthyridin-4-amine ethanesulfonate monohydrate was then dissolved in the solution.
Step 2: Edetate disodium was dissolved in water (approximately two fifths of the total amount used to achieve the final wt % in the gel).
Step 3: Carbomer 974P, xanthan gum, the solution from step 2, and water (approximately three fifths of the total amount used to achieve the final wt % in the gel) were added sequentially to the solution from Step 1 while mixing and mixing was continued until the thickeners were completely hydrated.
Step 4: Tromethamine was dissolved in water (20% by weight tromethamine) and the solution was added to the gel from step 3 while mixing. Mixing was continued until the gel was uniform. After mixing was complete the pH was measured.

TABLE 21

| Ingredient | Example 37 Gel (% w/w) |
|---|---|
| Drug (ethanesulfonate monohydrate) | 2.25 |
| Carbomer 974P | 2.00 |
| Xanthan gum | 1.00 |
| Propylene glycol | 40.00 |
| Methylparaben | 0.15 |
| Propylparaben | 0.03 |
| Edetate disodium | 0.05 |
| Tromethamine | 0.55 |
| Purified water | 53.97 |
| pH | 3.4 |
| Viscosity (cps) | 13,000 |

The complete disclosures of the patents, patent documents, and publications cited herein are incorporated by reference in their entirety as if each were individually incorporated. Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

1. An aqueous gel comprising:
water;
1-(2-methylpropyl )-1H-imidazo[4,5-c][1,5]naphthyridin-4-amine;
a pharmaceutically acceptable acid;
at least 10 wt-% of a water-miscible cosolvent; and
a thickener system comprising a negatively charged thickener;
wherein the aqueous gel has a viscosity of at least 1000 cps at 20° C.

2. An aqueous gel prepared by a method comprising combining components comprising:
water;
a 1-(2-methylpropyl)-1H-imidazo[4,5-c][1,5]naphthyridin-4-amine salt;
at least 10 wt-% of a water-miscible cosolvent; and
a thickener system comprising a negatively charged thickener;
wherein the aqueous gel has a viscosity of at least 1000 cps at 20° C.

3. The aqueous gel of claim 1, wherein the pharmaceutically acceptable acid is present in a stoichiometric amount relative to the 1-(2-methylpropyl)-1H-imidazo[4,5-c][1,5]naphthyridin-4-amine.

4. The aqueous gel of claim 1, wherein the 1-(2-methylpropyl)-1H-imidazo[4,5-c][1,5]naphthyridin-4-amine is provided as a salt.

5. The aqueous gel of claim 1, wherein the pharmaceutically acceptable acid is selected from the group consisting of an alkylsulfonic acid, a carboxylic acid, a halo acid, sulfuric acid, phosphoric acid, a dicarboxylic acid, a tricarboxylic acid, and combinations thereof.

6. The aqueous gel of claim 5, wherein the acid is selected from the group consisting of acetic acid, hydrobromic acid, D-gluconic acid, L-lactic acid, methanesulfonic acid, ethanesulfonic acid, propionic acid, and combinations thereof.

7. The aqueous gel of claim 2, wherein the 1-(2-methylpropyl)-1H-imidazo[4,5-c][1,5]naphthyridin-4-amine salt is a salt of an acid selected from the group consisting of an alkylsulfonic acid, a carboxylic acid, a halo acid, sulfuric acid, phosphoric acid, a dicarboxylic acid, a tricarboxylic acid, and combinations thereof.

8. The aqueous gel of claim 7, wherein the 1-(2-methylpropyl)-1H-imidazo[4,5-c][1,5]naphthyridin-4-amine salt is a salt of an acid selected from the group consisting of acetic acid, hydrobromic acid, D-gluconic acid, L-lactic acid, propionic acid, and combinations thereof.

9. The aqueous gel of claim 7, wherein the 1-(2-methylpropyl)-1H-imidazo[4,5-c][1,5]naphthyridin-4-amine salt is an alkylsulfonate salt.

10. The aqueous gel of claim 9, wherein the 1-(2-methylpropyl)-1H-imidazo[4,5-c][1,5]naphthyridin-4-amine alkylsulfonate salt is 1-(2-methylpropyl)-1H-imidazo[4,5-c][1,5]naphthyridin-4-amine ethanesulfonate or 1-(2-methylpropyl)-1H-imidazo[4,5-c][1,5]naphthyridin-4-amine methanesulfonate.

11. The aqueous gel of claim 1, wherein the 1-(2-methylpropyl)-1H-imidazo[4,5-c][1,5]naphthyridin-4-amine is present in an amount of at least 0.0001 wt-%, based on the total weight of the aqueous gel.

12. The aqueous gel of claim 1, wherein the water-miscible cosolvent is present in an amount of greater than 25 wt-%, based on the total weight of the aqueous gel.

13. The aqueous gel of claim 12, wherein the water-miscible cosolvent is present in an amount of at least 30 wt-%, based on the total weight of the aqueous gel.

14. The aqueous gel of claim 1, wherein the water-miscible cosolvent is present in an amount of no greater than 90 wt-%, based on the total weight of the aqueous gel.

15. The aqueous gel of claim 1, wherein the water-miscible cosolvent is selected from the group consisting of monopropylene glycol, dipropylene glycol, hexylene glycol, butylene glycol, glycerin, polyethylene glycol, diethylene glycol monoethyl ether, and combinations thereof.

16. The aqueous gel of claim 15, wherein the water-miscible cosolvent comprises monopropylene glycol.

17. The aqueous gel of claim 1, wherein the thickener system comprises at least two negatively charged thickeners of differing charge density.

18. The aqueous gel of claim 1, wherein the thickener system comprises at least two negatively charged thickeners selected from the group consisting of a cellulose ether, a polysaccharide gum, an acrylic acid polymer, and combinations thereof.

19. The aqueous gel of claim 18, wherein the thickener system comprises a polysaccharide gum and an acrylic acid polymer.

20. The aqueous gel of claim 19, wherein a weight ratio of the polysaccharide gum to the acrylic acid polymer is 1:20 to 20:1.

21. The aqueous gel of claim 1, wherein the thickener system comprises at least two negatively charged thickeners, each including carboxylic acid and/or carboxylate groups.

22. The aqueous gel of claim 21, wherein the thickener system comprises at least two negatively charged thickeners selected from the group consisting of carboxymethylcellulose sodium, xanthan gum, an acrylic acid polymer, and combinations thereof.

23. The aqueous gel of claim 1, wherein the thickener system is present in an amount of at least 0.1 wt-%, based on the total weight of the aqueous gel.

24. The aqueous gel of claim 1, wherein the thickener system is present in an amount of no greater than 7 wt-%, based on the total weight of the aqueous gel.

25. The aqueous gel of claim 1, wherein water is present in an amount of at least 10 wt-%, based on the total weight of the aqueous gel.

26. The aqueous gel of claim 1, wherein water is present in an amount of no greater than 95 wt-%, based on the total weight of the aqueous gel.

27. The aqueous gel of claim 1, further comprising a pharmaceutically acceptable pH adjusting agent.

28. The aqueous gel of claim 27, wherein the pharmaceutically acceptable pH adjusting agent is selected from the group consisting of hydrochloric acid, sodium hydroxide, tromethamine, potassium hydroxide, and combinations thereof.

29. The aqueous gel of claim 1 having a pH of 2 to 5.

30. The aqueous gel of claim 29 having a pH of 3 to 4.

31. The aqueous gel of claim 1 further comprising a preservative.

32. The aqueous gel of claim 31, wherein the preservative is selected from the group consisting of quaternary ammonium compounds, benzethonium chloride, parabens, boric acid, isothiazolinone, organic acids, alcohols, carbamates, chlorhexidine, and combinations thereof.

33. The aqueous gel of claim 32, wherein the preservative is selected from the group consisting of methylparaben, propylparaben, and combinations thereof.

34. The aqueous gel of claim 31, wherein the preservative is present in an amount of at least 0.005 wt-%, based on the total weight of the aqueous gel.

35. The aqueous gel of claim 31, wherein the preservative is present in an amount of no greater than 1.0 wt-%, based on the total weight of the aqueous gel.

36. The aqueous gel of claim 1 further comprising a chelating agent.

37. The aqueous gel of claim 36, wherein the chelating agent is selected from the group consisting of ethylenediaminetetracetic acid, ethylenediaminetetracetic acid disodium salt, ethylenediaminetetracetic acid disodium salt dihydrate, and combinations thereof.

38. The aqueous gel of claim 37, wherein the chelating agent is ethylenediaminetetracetic acid disodium salt dihydrate.

39. The aqueous gel of 36, wherein the chelating agent is present in an amount of at least 0.001 wt-%, based on the total weight of the aqueous gel.

40. The aqueous gel of claim 36, wherein the chelating agent is present in an amount of no greater than 2.0 wt-%, based on the total weight of the aqueous gel.

41. The aqueous gel of claim 1, wherein the negatively charged thickener is not covalently bonded to the 1-(2-methylpropyl )-1H-imidazo[4,5-c][1,5]naphthyridin-4-amine.

42. A method of delivering 1-(2-methylpropyl)-1H-imidazo[4,5-c][1,5]naphthyridin-4-amine to a mucosal tissue of a subject, the method comprising applying the aqueous gel of claim 1 to the mucosal tissue of the subject.

43. The method of claim 42, wherein the mucosal tissue is associated with a condition selected from the group consisting of a cervical dysplasia, a papilloma virus infection of the cervix, a low-grade squamous intraepithelial lesion, a high-grade squamous intraepithelial lesion, atypical squamous cells of undetermined significance, a cervical intraepithelial neoplasia, an atopic allergic response, allergic rhinitis, a neoplastic lesion, and a premalignant lesion.

44. The method of claim 43, wherein the mucosal tissue is on a cervix of the subject and the associated condition is selected from the group consisting of cervical dysplasia, high-grade squamous intraepithelial lesions, low-grade squamous intraepithelial lesions, and atypical squamous cells of undetermined significance with the presence of high risk HPV.

45. The method of claim 44, wherein the mucosal tissue is on the cervix of the subject and the associated condition is atypical squamous cells of undetermined significance with the presence of high risk HPV.

46. The method of claim 43, wherein the mucosal tissue is on the cervix of the subject and the associated condition is a papilloma virus infection of the cervix.

47. The method of claim 42, wherein the aqueous gel is applied to the mucosal tissue of the subject using a device selected from the group consisting of a barrel type applicator, a tampon, a cervical cap, a diaphragm, a cotton swab, a cotton sponge, a foam sponge, and a suppository.

48. The method of claim 47, wherein the device is a barrel type applicator.

49. The method of claim 48, wherein the barrel type applicator is prefilled.

50. A kit comprising a barrel type applicator and the aqueous gel of claim 1.

51. The kit of claim 50, wherein the kit further includes a container, that is separate from the applicator and that includes the aqueous gel.
